(19) United States
(12) Patent Application Publication
(10) Pub. No.: US 2012/0015903 A1
(43) Pub. Date: Jan. 19, 2012

(54) PHARMACEUTICAL COMPOSITION FOR INHIBITING INCREASE OF IgA

(75) Inventors: WEN-MIEN WU, TAIPEI COUNTY (TW); SU-SHAN YU, TAIPEI COUNTY (TW)

(73) Assignee: FU JEN CATHOLIC UNIVERSITY, TAIPEI COUNTY (TW)

(21) Appl. No.: 12/954,776

(22) Filed: Nov. 26, 2010

(30) Foreign Application Priority Data
Jul. 16, 2010 (TW) 099123590

(51) Int. Cl. A61K 31/716; A61P 19/00; A61P 13/12

(52) U.S. Cl. 514/54

(57) ABSTRACT

The present invention discloses a pharmaceutical composition for inhibiting increase of immunoglobulin A (IgA) comprising β-glucan and a pharmaceutical acceptable carrier. The β-glucan is extracted from Saccharomyces Cerevisiae, and the effective dose of the β-glucan is 300-450 mg/70 kg of body weights/per day, preferably, 350-400 mg/70 kg of body weights/day.

### FIELD OF THE INVENTION

The present invention relates to a pharmaceutical composition; and more particularly, to a pharmaceutical composition for inhibiting an increase of immunoglobulin A (IgA).

### BACKGROUND OF THE INVENTION

The symptoms of many diseases, such as immunoglobulin A (IgA) nephropathy or ankylosing spondylitis (AS) and so on, have an abnormal increase in IgA. The IgA nephropathy is the most common disease in glomerulonephritis. So far, the etiology thereof is not clear, but medical studies have found that about 15% patients with the ankylosing spondylitis will have a complication, IgA nephropathy. The IgA nephropathy patients can be found in excessive IgA. IgA and some kinds of complement proteins in the immune system will form an immune complex. The immune complex will be precipitated in the glomeruli to result in a series of injuries. Most of the IgA nephropathy is primary kidney inflammation, and about 60% patients, the IgA concentration thereof is higher than that of normal persons.

Up to now, there is no perfect therapy in the IgA nephropathy treatment, and the therapeutic approach thereof may be a supportive therapy and an immunosuppressive therapy of controlling immune and inflammatory response. For the supportive therapy, the blood pressure of the patients may be decreased by taking angiotensin-converting enzyme inhibitors and angiotensin-II receptor blockers thereby moderating the renal impairment.

The purpose of the immunosuppressive therapy is to inhibit the progress of the nephropathy involved of IgA immunoglobulin, and to moderate the renal impairment. The first-line drug of the immunosuppressive therapy is usually corticosteroid applicable to patients with still retaining normal renal functions. The second-line drugs may be cytotoxic drugs and immunosuppressive agents, which are usually for mixed use and applicable to patients with the renal impairment. However, the pharmacological action of the immunosuppressive agents is non-specific reaction, and many side effects may be caused, such as decreasing immunity to induce severe infection; and anemia resulted from inhibiting bone marrow hematopoietic functions to decrease the counts of white blood cells and platelets. After taking the immunosuppressive agents for a long time, cancers may be caused, female patients may conceive difficulty, and the fetus may be malformation for the pregnant women. Furthermore, the quantity and quality of sperms may be reduced for males. Because the pharmacological action of the immunosuppressive agents is non-specific reaction, the immune functions of the patients may be reduced drastically to lead to increase pathogenic microorganism infections.

### SUMMARY OF THE INVENTION

In view of the aforementioned drawbacks in prior art, an object of the present invention is to provide a pharmaceutical composition for inhibiting an increase of immunoglobulin A (IgA) so as to resolve the problem of reducing drastically immune functions caused by taking the immunosuppressive agents for a long time.

According to the object of the present invention, the pharmaceutical composition for inhibiting the increase of IgA comprises β-glucan and a pharmaceutical acceptable carrier. The β-glucan may be extracted from Saccharomyces Cerevisiae. The pharmaceutical acceptable carrier may be selected from the group consisting of a flavoring agent, a sweetener, a preservative, an anti-oxidative agent, a chelating agent, an osmotic agent, an lubricant, a tablet adjuvant, a coloring agent, a binding agent and a pharmaceutical compatible carrier to produce the pharmaceutical composition to a tablet form, a liquid form, a powder form or other dosage forms. The effective dose of the β-glucan is in a range of 300 to 450 mg/70 kg of body weight/day, and preferably, in a range of 350 to 400 mg/70 kg of body weight/day.

Accordingly, the pharmaceutical composition for inhibiting the increase of IgA according to the present invention provides one or more of the following advantages:

(1) Under normal conditions, antibodies do not be influenced by taking the pharmaceutical composition of the present invention, i.e. IgA secretion does not be inhibited. However, when IgA is increased abnormally, the IgA concentrations can be effectively decreased by taking the pharmaceutical composition of the present invention.

(2) After patients take the pharmaceutical composition for a long time, side effects, such as decrease of immunity, inhibition of bone marrow hematopoietic functions or anemia, do not be caused.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 illustrates the effect of the pharmaceutical composition of the present invention on the levels of IgA in Peyer's patches (PP) supernatant after the mice are fed with the pharmaceutical composition of the present invention for two weeks;

FIG. 2 illustrates the effect of the pharmaceutical composition of the present invention on the levels of IgA in the PP supernatant after the mice are fed with the pharmaceutical composition of the present invention for six weeks;

FIG. 3 illustrates the effects of the pharmaceutical composition of the present invention on IgA concentrations in the PP supernatant;

FIG. 4 illustrates the effects of the pharmaceutical composition of the present invention on IgG concentrations in the PP supernatant

FIG. 5 illustrates the effects of the pharmaceutical composition of the present invention on IgM concentrations in the PP supernatant;

FIG. 6 illustrates the effect of the pharmaceutical composition of the present invention on transforming growth factor-β1 (TFG-β1) concentrations in the intestinal PP supernatant;

FIG. 7 illustrates the effect of the pharmaceutical composition of the present invention on interlukin-10 (IL-10) concentrations in the intestinal PP supernatant;

FIG. 8 illustrates the effect of the pharmaceutical composition of the present invention on TGF-β1 in the mesenteric lymph node (MLN) supernatant; and

FIG. 9 illustrates the effect of the pharmaceutical composition of the present invention on IL-10 in the MLN supernatant.

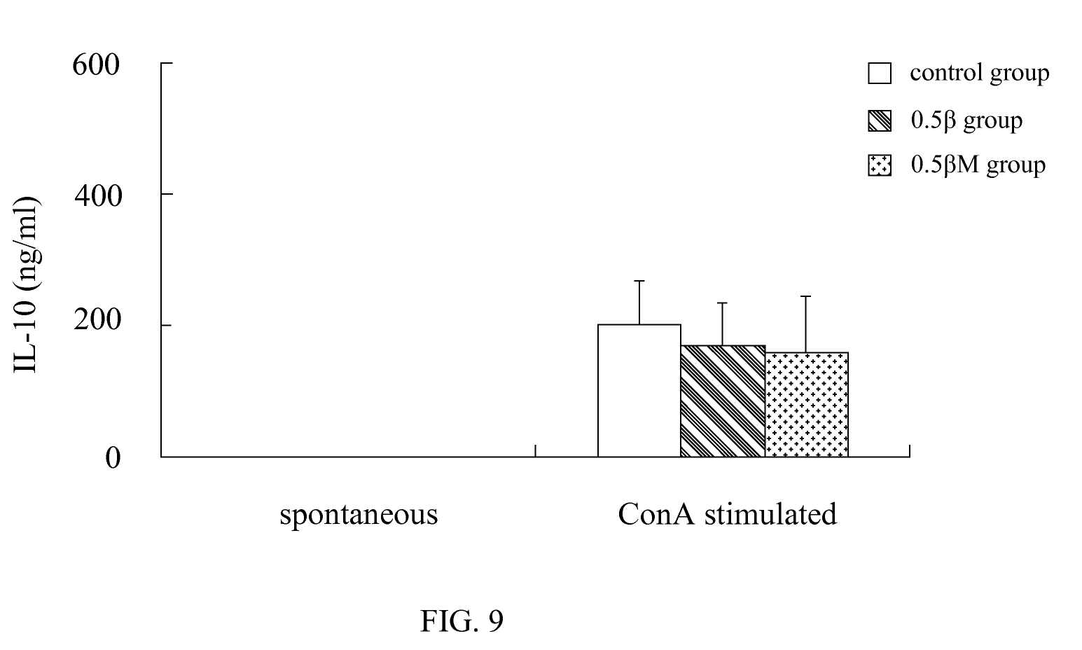

### DETAILED DESCRIPTION OF THE PREFERRED EMBODIMENTS

The present invention will now be described with some preferred embodiments thereof with reference to the accompanying drawings. It is understood the experimental data shown in the embodiments are provided only for easy interpretation of the technical means of the present invention and should in no means be considered as restriction to the present invention.

### Embodiment 1

### The Compose of the Pharmaceutical Composition

A pharmaceutical composition for inhibiting the increase of IgA comprises β-glucan and a pharmaceutical acceptable carrier. The β-glucan may be extracted from Saccharomyces Cerevisiae. The pharmaceutical acceptable carrier may be selected from the group consisting of a flavoring agent, a sweetener, a preservative, an anti-oxidative agent, a chelating agent, an osmotic agent, an lubricant, a tablet adjuvant, a coloring agent, a binding agent and a pharmaceutical compatible carrier to produce the pharmaceutical composition to a tablet form, a liquid form, a powder form or other dosage forms. The effective dose of the β-glucan is in a range of 300 to 450 mg/70 kg of body weight/day, and preferably, in a range of 350 to 400 mg/70 kg of body weight/day.

### Embodiment 2

### Preferable Embodiment

Animal Experiment

In the present embodiment, the β-glucan in the pharmaceutical composition of the present invention is purified from the cell wall of saccharomyces cerevisiae. The experimental animals are BALB/c female mice, which are randomly classified into four groups, a control group, a 0.1β group, a 0.5β group and a 0.5βM group. There are five mice in each group, and the 0.1β group, the 0.5β group and the 0.5βM group are classified according to the concentration of the β-glucan. The concentrations of the β-glucan in the 0.1β group and 0.5β group are 0.39 and 1.95 mg/ml, respectively. The mice of the 0.5βM group are fed with β-glucan mixed with milk powder, and the concentration of the β-glucan thereof is 1.95 mg/ml. The mice of the control group are fed with distilled water. The volume of gastric feeding is 0.5 ml in each mouse every time. After feeding two or six weeks, Peyer's patches (PP) and mesenteric lymph nodes (MLN) are obtained from each mouse to cell culture.

After two or six weeks, the peritonea of the mice are cut to pick out the small intestine and slightly spread thereby finding the transparent mesentery and the capillary collecting site near the cecum, and that is the mesenteric lymph node. Furthermore, the Peyer's patch is an elliptical lymph node protruding on the small intestinal wall, looks like a yellow rice shape, and is distributed from the duodenum to the cecum under the stomach.

Additionally, all bellow experimental data are represented by mean±standard deviation (SD), and the statistical analysis is analyzed via Microsoft® Office Excel 2003. The results of the 0.1β group, the 0.5β group and the 0.5βM group compared with the control group are analyzed by student's t-test. When p<0.05, there is a significant difference between two groups and the significant difference is represent by “*” in the figures.

The effect of the pharmaceutical composition of the present invention on IgA antibodies

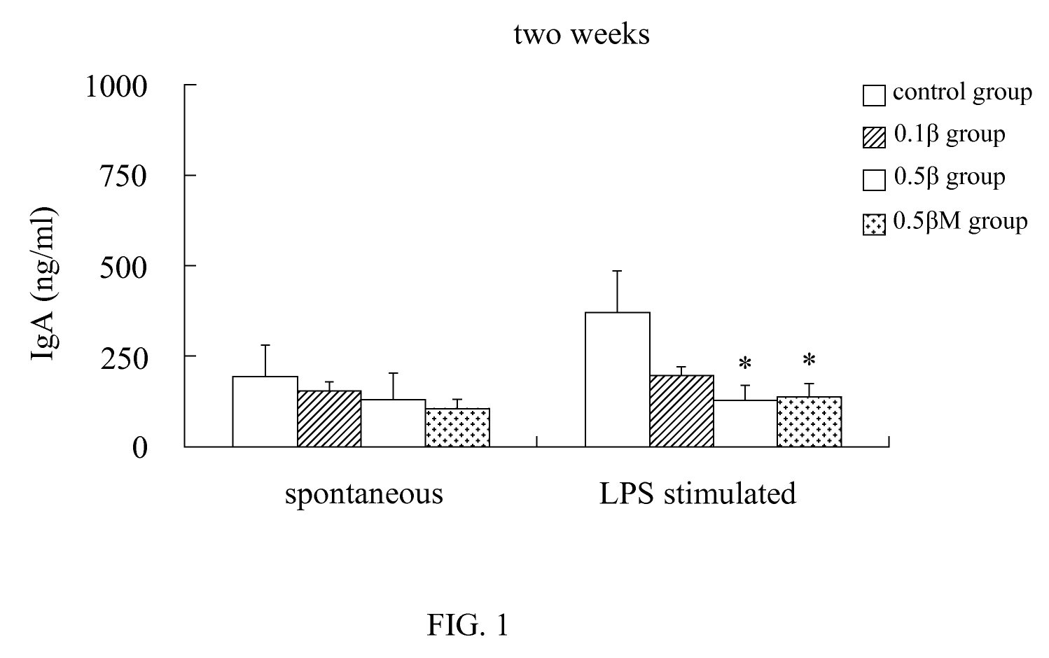

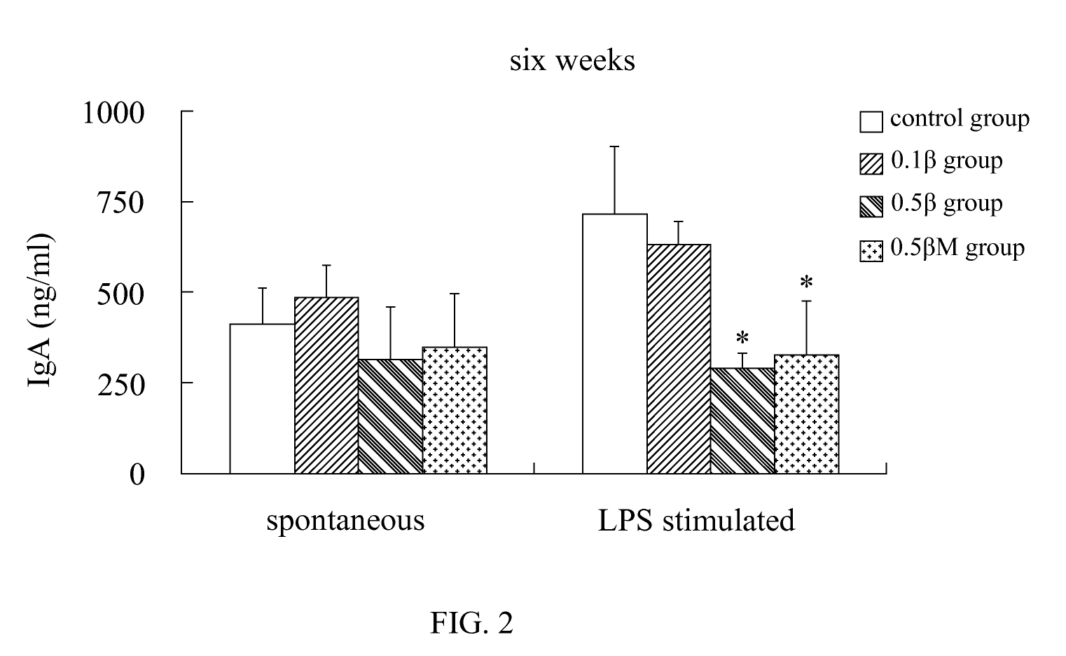

Because the PP cells are composed of B cells and IgA is secreted from the B cells, the IgA concentration in the supernatant of the PP cells is determined in the present embodiment. After the mice are fed with the pharmaceutical composition of the present invention with different β-glucan concentrations for two or six weeks, the mice are sacrificed to obtain PP cells. The PP cells are treated with lipopolysaccharide (LPS) for seventy-two hours, and then the IgA concentration of the PP supernatant is measured. The results show that the IgA concentrations of the PP supernatant in the 0.5β group and the 0.5βM group are significantly lower than that in the control group after feeding two or six weeks, as shown in FIG. 1 and FIG. 2, respectively. However, in the PP supernatant without treating with the LPS, the result of the IgA concentrations in each group comparing with the control group do not have the significant difference. Accordingly, after the mice are fed with the pharmaceutical composition of the present invention for two weeks, the concentrations of IgA secreted from the PP cells can be reduced under treating with the LPS. Furthermore, no matter for feeding the pharmaceutical composition of the present invention two or six weeks without treating with the LPS, the ability of secreting IgA of the PP cells is not affected by the pharmaceutical composition of the present invention.

The effect of the pharmaceutical composition of the present invention on intestinal antibodies

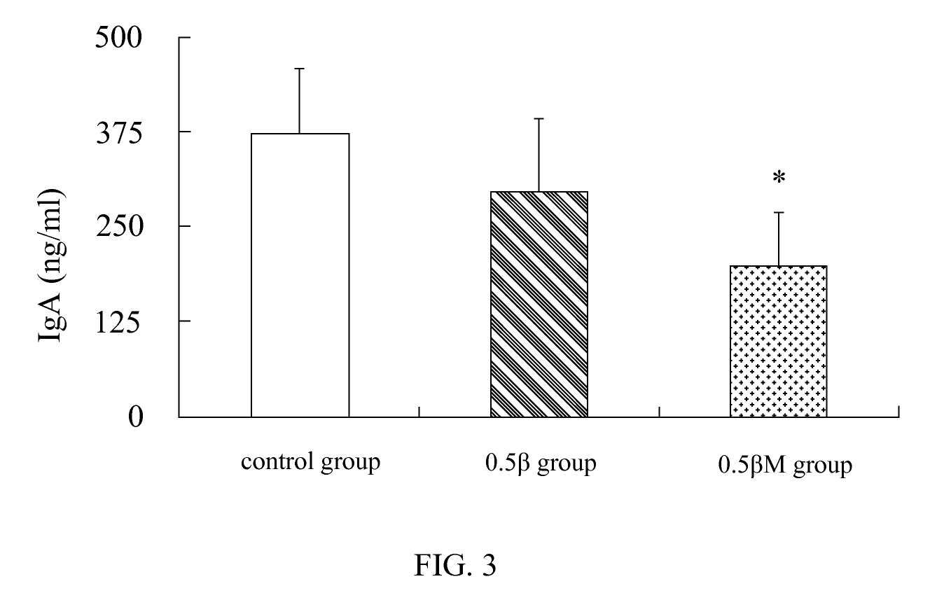

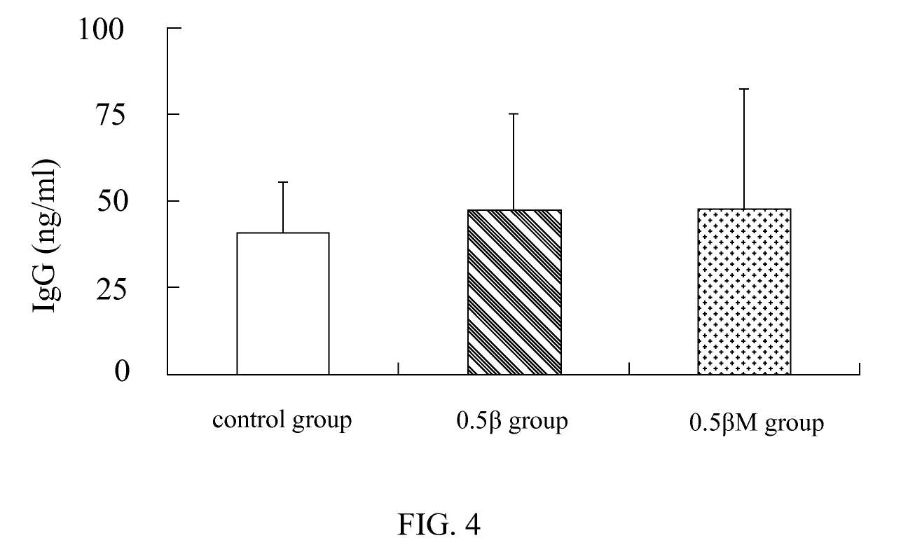

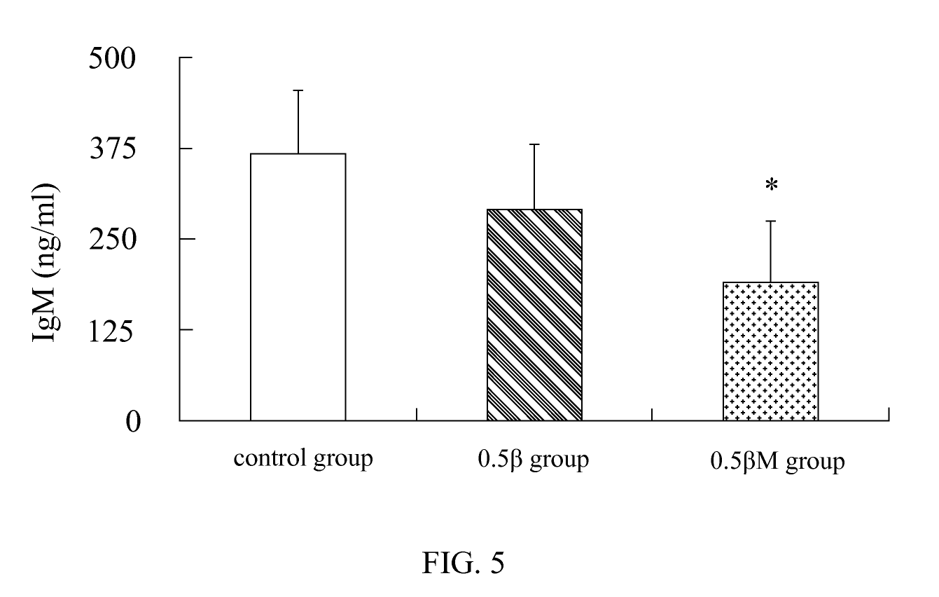

Please refer to FIG. 3 to FIG. 5, those are the effects of the pharmaceutical composition of the present invention on IgA, IgG and IgM concentrations respectively in the PP supernatant. As shown, the IgA and IgM levels of the PP supernatant with LPS in the 0.5βM group are significantly lower than those in the control group. The IgA and IgM levels of the PP supernatant with LPS in the 0.5β group are lower than those in the control group, but no statistical difference is found. The IgG contents among the control group, the 0.5β group and the 0.5βM group are not significantly different. As above results, the mice fed with 1.95 mg/ml β-glucan of the present invention for two weeks have the inhibition on the IgA and IgM secretion in the PP supernatant, but have no effect on IgG secretion. Compared with the secretion concentrations of three antibodies, it is to find out the account of the IgG secreted from the PP cells is a very small number, which is indicated that IgG is not a main antibody form in this immune system. Therefore, the results show that the pharmaceutical composition of the present invention has the ability of decreasing the antibody secretion while treating with LPS.

The effect of the pharmaceutical composition of the present invention on cytokine levels in the PP supernatant

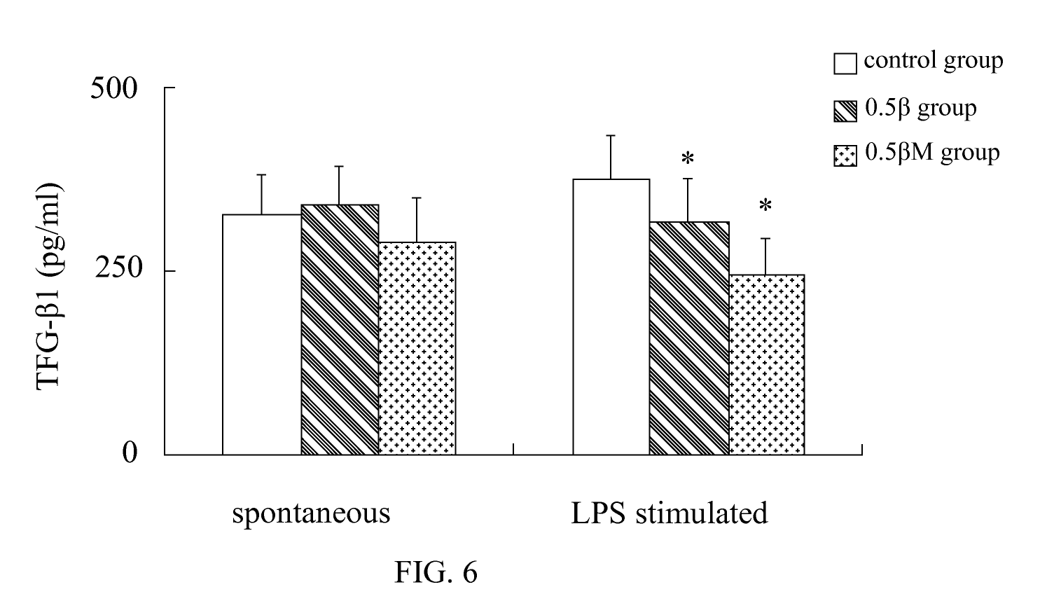

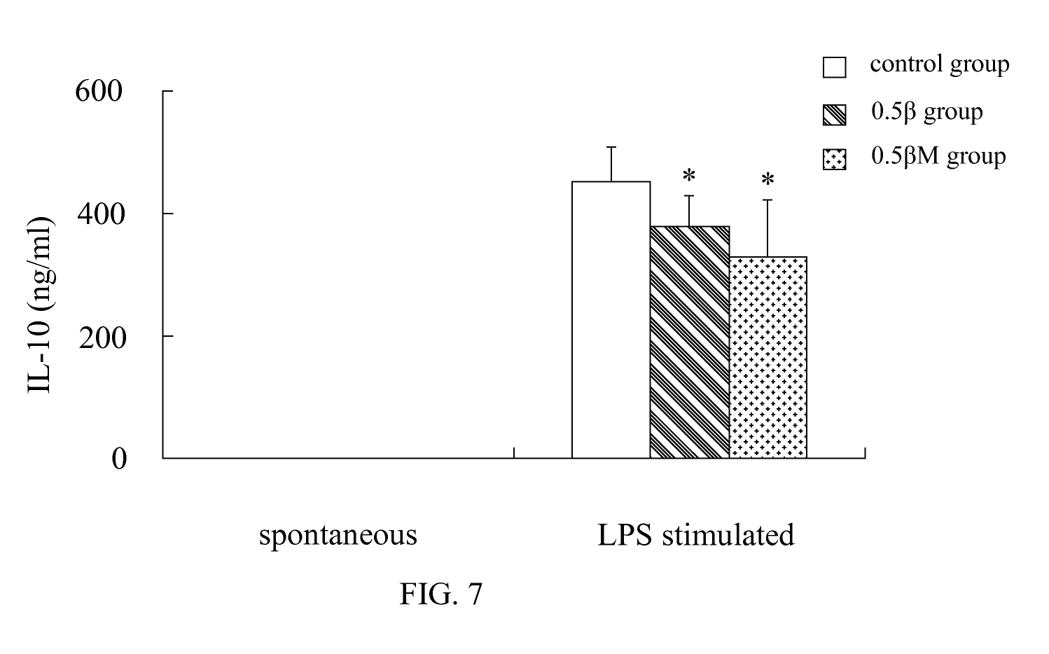

Although B cells are major cell populations in Peyer's patches, other cells, such as dentritic cells (DC) or T cells, as well as participate in the activation and maturation of the B cells via secreting cytokines. For example, the transforming growth factor-β1 (TFG-β1) can induce IgA transformation, and interlukin-10 (IL-10) can promote IgA secretion. Thus, in the present embodiment, the levels of TFG-β1 and IL-10 in the PP supernatant of the mice fed with 1.95 mg/ml β-glucan of the pharmaceutical composition of the present invention for two weeks are determined. The results show the levels of TFG-β1 and IL-10 in the 0.5β group and 0.5βM group are both lower than that in the control group under treating with LPS, as shown in FIG. 6 and FIG. 7. Additionally, without treating with LPS, PP cells have high TFG-β1 expression and the levels of IL-10 are not found. It may be that the active and inactive TFG-β1 is existed in the cell supernatant, and the samples have to acid hydrolysis in the measuring procedure, such that the obtained data are the sum of the active and inactive TFG-β1.

The effect of the pharmaceutical composition of the present invention on cytokine levels in the MLN supernatant

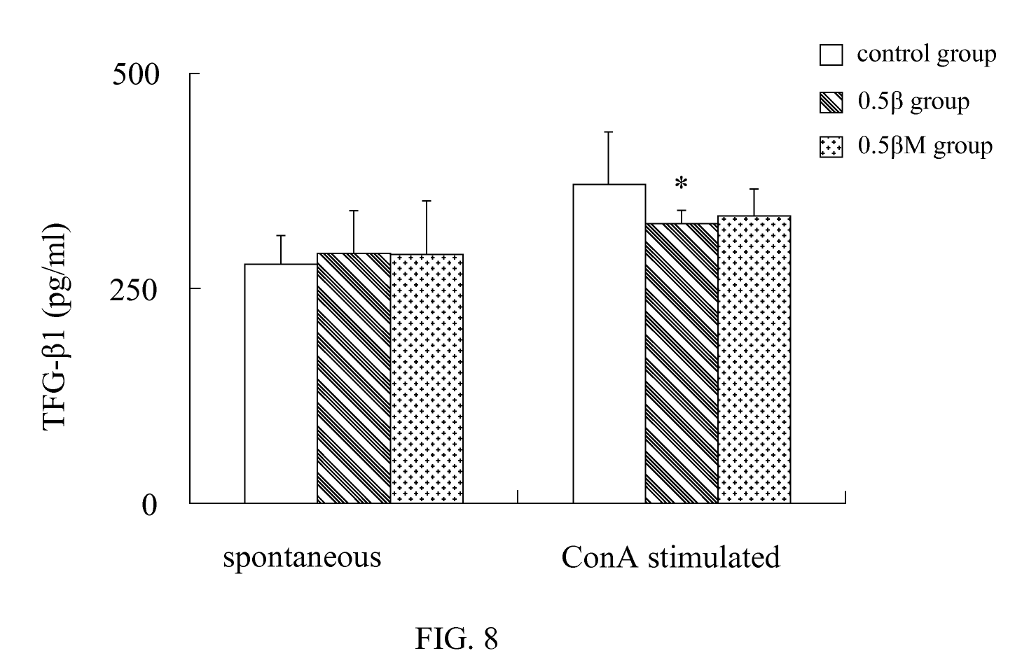

In factors affecting IgA secretion, in addition to the cytokines secreted from the PP cells, the related conversion cytokines secreted from T cells are also important regulatory factors. The intestinal T cells are mainly distributed in the MLNs, and therefore, the MLNs of the mice fed with 1.95 mg/ml β-glucan of the pharmaceutical composition of the present invention for two weeks are treated with concanavalin A (ConA) for 48 hours. Further, the levels of TFG-β1 and IL-10 in the MLN supernatant are measured. The results show that the secretion levels of TFG-β1 in the 0.53 group are significantly lower than that in the control group, and there is a downward trend in the secretion levels of TFG-β1 of the 0.5βM group, as shown in FIG. 8. The levels of IL-10 in the 0.5β group and the 0.5βM group have a downward trend compared with those in the control group. According to the above results, the levels of TFG-β1 in the PP cells are similar with that in the MNL cells. Compared with the PP cells, the secretion ability of IL-10 of the MNL cells is weaker, and thus it is not significant decrease on the levels of IL-10 in the MNL cells.

To sum up above results, the effective dose of β-glucan in the pharmaceutical composition of this invention is converted from the dose of the aforementioned animal experiment. The pharmaceutical composition of the present invention just has the ability of inhibiting IgA secretion while treating with foreign materials, such as LPS or ConA. Under normal conditions, the IgA concentrations are not influenced by the pharmaceutical composition of the present invention. On the other hand, when the PP cells and the MNL cells are treated with LPS or ConA, the secretion levels of TGF-β1 and IL-10 are absolutely decreased by the pharmaceutical composition of the present invention. Additionally, all antibodies maintain the balance status without treating with any foreign materials. Therefore, the pharmaceutical composition of the present invention does not affect the immune response under normal conditions. When IgA concentration are increased abnormally, such as suffering from IgA nephropathy or ankylosing spondylitis, the pharmaceutical composition of the present invention has the ability of inhibiting IgA secretion and can regulate relating-antibodies about IgA secretion.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A pharmaceutical composition for inhibiting increase of immunoglobulin A, comprising:
β-glucan; and
a pharmaceutical acceptable carrier.

2. The pharmaceutical composition as claimed in claim 1, wherein the β-glucan is extracted from Saccharomyces Cerevisiae.

3. The pharmaceutical composition as claimed in claim 1, wherein the pharmaceutical acceptable carrier is selected from the group consisting of a flavoring agent, a sweetener, a preservative, an anti-oxidative agent, a chelating agent, an osmotic agent, an lubricant, a tablet adjuvant, a coloring agent, a binding agent and a pharmaceutical compatible carrier.

4. The pharmaceutical composition as claimed in claim 1, wherein a dosage form of the pharmaceutical composition comprises a tablet form, a liquid form or a powder form.

5. The pharmaceutical composition as claimed in claim 1, wherein an effective dose of the β-glucan is in a range of 300 to 450 mg/70 kg of body weight/day.

6. The pharmaceutical composition as claimed in claim 1, wherein an effective dose of the β-glucan is in a range of 350 to 400 mg/70 kg of body weight/day.
